(19) United States
(12) Patent Application Publication
(10) Pub. No.: US 2007/0287672 A1
(43) Pub. Date: Dec. 13, 2007

(54) Macromolecular Gsh-Activiated Glyoxylase I Inhibitors

(76) Inventors: Donald Creighton, Catonsville, MD (US); Donald J. Creighton, Catonsville, MD (US); Zhe-Bin Zheng, Xinghuang Minhang (CN)

Correspondence Address: SUGHRUE MION, PLLC, 2100 PENNSYLVANIA AVENUE, N.W., SUITE 800, WASHINGTON, DC, 20037, US

(21) Appl. No.: 11/587,160

(22) PCT Filed: Apr. 20, 2005
(86) PCT No.: PCT/US05/13361

§ 371 Date: Jul. 10, 2007

Related U.S. Application Data
(60) Provisional application No. 60/563,864, filed on Apr. 20, 2004.

(51) Int. Cl. C07C 333/00; A61K 31/10; A61K 38/06; C07C 50/22; C08F 2/06; C12N 5/00; C07K 5/08; C07C 49/543; A61P 35/00

(52) U.S. Cl. 514/18; 435/375; 514/708; 526/75; 530/331; 552/200; 552/261; 568/37

(57) ABSTRACT

This invention relates to macromolecular prodrugs having antitumor activity when activated by GSH, and methods for synthesizing and administering these to patients. More particularly, this invention relates to, inter alia, the synthesis and use of polyacrylamide carriers to target anticancer prodrugs to tumors, and to release active antitumor agents selectively in tumor cells. These active antitumor agents target the active site of the methylglyoxal-detoxifying enzyme glyoxalase I to thereby cause tumor regression.

### CROSS-REFERENCE TO RELATED APPLICATION

This application claims benefit of U.S. Provisional Application No. 60/563,864 filed Apr. 20, 2004; the disclosure of which is incorporated herein by reference.

### GOVERNMENT RIGHTS

This invention was created in part using funds from the federal government under a grant from the National Cancer Institute (CA 59612) and under a grant from the Department of Defense (DAMD17-99-1-9275). The United States Government, therefore, has certain rights in this invention.

### FIELD OF INVENTION

This invention relates to macromolecular prodrugs having antitumor activity when activated by GSH, and methods involving administering these to patients. More particularly, this invention relates to, inter alia, the synthesis and use of polyacrylamide carriers to target anticancer prodrugs to tumors, and to release active antitumor agents selectively in tumor cells. These active antitumor agents target the active site of the methylglyoxal-detoxifying enzyme glyoxalase I, or alkylate proteins and polynucleic acids critical to cell viability. Either one or both of which will cause tumor regression.

### BACKGROUND OF THE INVENTION

Recent advances in understanding the metabolism of methylglyoxal in mammalian cells suggest that the glutathione (GSH)-dependent glyoxalase enzyme system is a useful target for antitumor drug development (Creighton et al, Drugs of the Future, 25:385-392 (2000)). The physiological function of this detoxification pathway is to remove cytotoxic methylglyoxal from cells as D-lactate via the sequential action of the isomerase glyoxalase I (GlxI) and the thioester hydrolase glyoxalase II (GlxII), as shown in Scheme 1 below (Creighton et al, “Glutathione-Dependent Aldehyde Oxidation Reactions”, In Molecular Structure and Energetics: Principles of Enzyme Activity, Liebman et al, Eds.; VCH Publishers, 9: 353-386 (1988)).

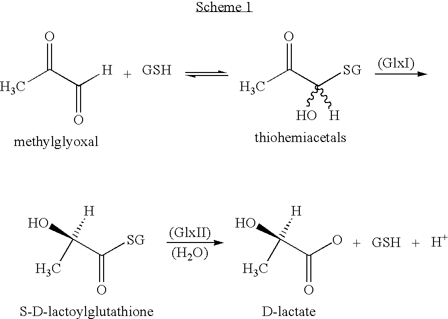

Methylglyoxal is a highly reactive alpha-ketoaldehyde that arises as a normal by-product of carbohydrate metabolism (Richard et al, Biochemistry, 30:4581-4585 (1991)) and is capable of covalently modifying proteins and nucleic acids critical to cell viability (Reiffen et al, J. Cancer Res. Clin. Oncol., 107:206-219 (1984); Ayoub et al, Leuk. Res., 17:397-401 (1993); Baskaran, et al, Biochem. Int., 212:166-174 (1990); Ray et al, Int. J. Cancer, 47:603-609 (1991); White et al, Chem-Biol. Interact., 38:339-347 (1982); and Papoulis et al, Biochemistry, 34:648-655 (1995)).

Inhibitors of GlxI have been investigated as anticancer agents because of their potential to induce elevated concentrations of methylglyoxal in tumor cells (Creighton et al (2000), supra), and because of the observation that rapidly dividing tumor cells are exceptionally sensitive to the cytotoxic effects of exogenous methylglyoxal (Ray et al, supra; White et al, supra; and Papoulis et al, supra). While the basis of this sensitivity is not well understood, it appears to arise, in part, from methylglyoxal induced activation of the stress-activated protein kinases c-Jun NH₂-terminal kinase 1 (JNK1) and p38 mitogen-activated protein kinase (MAPK), which in turn leads to caspase activation and apoptosis (programmed cell death) in tumor cells (Sakamoto et al, Clinical Cancer Research, 7:2513-2518 (2001); and Sakamoto et al, J. Biol. Chem., 277:45770-45775 (2002)). Moreover, since methylglyoxal is able to covalently modify nucleotide bases in DNA (Papoulis et al, supra), methylglyoxyl is probably genotoxic as well.

Of particular interest are inhibitors of GlxI that are hydrolytically destroyed by the thioester hydrolase GlxII, which then offer a selective strategy for specifically inhibiting tumor cells, as normal cells contain much higher concentrations of GlxII than tumor cells. Table 1 below shows a comparison of the activities of GlxI and GlxII in normal versus cancer cells (Creighton et al (2000), supra).

TABLE 1
Reported Glyoxalase Activities in
Normal Cells versus Cancer Cells

| | Glyoxalase Activity | | |
|---|---|---|---|
| | (mU/mg protein) | | |
| Tissue | GlxI | GlxII | GlxI/GlxII |

Normal

| | | | |
|---|---|---|---|
| brain (human) | 1113 ± 19 | 817 ± 156 | 1.4 |
| liver (human) | 209 ± 56 | 360 ± 13 | 0.6 |
| heart (hamster) | 339 ± 24 | 280 ± 47 | 1.2 |
| kidney (human) | 323 ± 48 | 330 ± 86 | 1.0 |
| lymphocytes (mouse) | 360 ± 30 | 200 ± 30 | 1.8 |

Tumor

| | | | |
|---|---|---|---|
| melanoma B16 (mouse) | 370 ± 160 | 66 ± 18 | 5.6 |
| leukemia L1210 (mouse) | 310 ± 30 | 20 ± 3 | 15.5 |
| glioblastoma (human) | 290 ± 56 | 53 ± 10 | 5.5 |
| fibroadenoma mammae (human) | 419 ± 73 | 27 ± 7 | 15.5 |
| bladder HT-1107 (human) | 542 ± 38 | 8 ± 1 | 67.8 |
| prostate PC-3 (human) | 4206 ± 294 | 45 ± 3 | 93.4 |
| testis T1 (human) | 4767 ± 275 | 94 ± 12 | 51.0 |
| colon HT29 (human) | 542 ± 59 | 11 ± 1 | 49.3 |

Thus, normal cells may be able to detoxify thioester inhibitors of hGlxI more rapidly than the corresponding tumor cells, resulting in higher steady-state concentrations of the inhibitors in tumor cells.

Consistent with this hypothesis, the diethylester prodrug form of S—(N-p-chlorophenyl-N-hydroxycarbamoyl)glutathione, CHG(Et)₂, which is both an inhibitor of hGlxI and a substrate for hGlxII (Murthy et al, J. Med. Chem., 37:2161 (1994)), is significantly more toxic to murine leukemia L1210 cells than to normal splenic lymphocytes in culture, reflecting, in part, the 10-fold lower activity of hGlxII in L1210 cells versus splenic lymphocytes (Kavarana et al, J. Med. Chem., 42:221-228 (1999)).

Further, an antitumor strategy targeting hGlxI provides benefits over more established chemotherapies that attack rapidly dividing tumor cells at various stages of mitosis, or that arrest tumor cells at some stage in the cell cycle. For example, many of the small molecule antitumor drugs currently in use target rapidly dividing tumor cells by either directly or indirectly inhibiting DNA and/or protein synthesis. Thus, these drugs will also adversely affect rapidly dividing normal cells, like those of the intestinal epithelium and bone marrow. As a result, side-effects of antitumor agents currently in use often include myelosuppression, intestinal disorders, dose-dependent cardiotoxicity, pulmonary fibrosis, anaphylactic reactions, alopetia, and anorexia.

A known class of transition state analogue inhibitors of human GlxI are S—(N-aryl/alkyl-N-hydroxycarbamoyl)glutathiones. These thioester derivatives of GSH mimic the stereoelectronic features of the tightly bound transition state species that flank the ene-diolate intermediate that forms along the reaction coordinate of the enzyme. As such, these compounds are the strongest known competitive inhibitors of hGlxI, with inhibition constants (Kᵢ) in the mid-nanomolar range: S—(N-p-chlorophenyl-N-hydroxycarbamoyl)glutathione (CHG), Kᵢ=46 nM; S—(N-p-bromophenyl-N-hydroxycarbamoyl)glutathione (BHG), Kᵢ=14 nM; S—(N-p-iodophenyl-N-hydroxycarbamoyl)glutathione (IHG), Kᵢ=0 nM; and S—(N-hexyl-N-hydroxycarbamoyl)glutathione, Kᵢ=16 nM (Kalsi et al, J. Med. Chem., 43:3981-3986 (2000)).

The transition state analogue inhibitors are also slow substrates for bovine liver GlxII, which suggests that these compounds may selectively inhibit tumor cells over normal cells (Murthy et al, supra).

The effectiveness of the transition state analogue inhibitors can be measured by their specificity for the GlxI active site and by the time they occupy the active site, thereby blocking access of the enzyme's natural substrate (GSH-methylglyoxal thiohemiacetal). An inhibitor with a low competitive inhibition constant (Kᵢ) associates with the active site of an enzyme with higher affinity and greater specificity, and therefore, occupies the active site for a longer period of time than inhibitors with higher Kᵢ values.

However, given the poor cell permeability of multiply charged GSH-based inhibitors, prodrug strategies have been investigated to enhance the cell permeability of these compounds.

For example, the transition state analogue inhibitors are lethal to different human and murine tumor cell lines in culture when administered as diethyl ester prodrugs (U.S. Pat. No. 5,616,563) (Kavarana et al, supra). After diffusion into cells, the prodrugs undergo esterase-catalyzed de-esterification inside the cell to give the di-acid form of the transition state analogue. The diethyl ester prodrugs of S—(N-phenyl-N-hydroxycarbamoyl)glutathione, PHG(Et)₂; S—(N-p-bromophenyl-N-hydroxycarbamoyl)glutathione, BHG(Et)₂; and S—(N-p-chlorophenyl-N-hydroxycarbamoyl)glutathione, CHG(Et)₂, inhibit the growth of murine leukemia L1210 cells in culture with IC₅₀ values of 63, 16, and 5 μM, respectively, after 72 hours of incubation.

A second strategy for delivering the transition state analogue inhibitors to tumors is as the membrane-permeable sulfoxide prodrug S—(N-4-chlorophenyl-N-hydroxycarbamoyl)ethyl sulfoxide, shown as (2) in scheme 2 below, which undergoes an acyl-interchange reaction with GSH to give CHG, shown as (1) in scheme 2 below (Hamilton et al, J. Med. Chem. 42:1823-1827 (1999)). 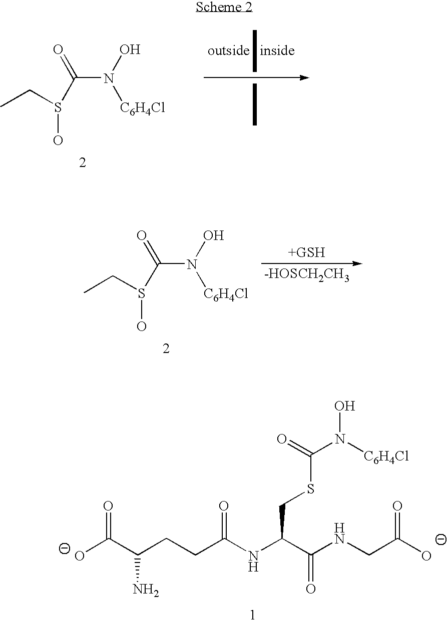
This prodrug is cytostatic and cytotoxic to several different tumor cell lines in vitro.

Also, electrophilic endocyclic enones, like 2-crotonyloxymethyl-2-cyclohexenone, shown as (3) in scheme 3 below, rapidly diffuse across cell membranes and undergo a Michael addition reaction with intracellular GSH to give the reactive exocyclic enone, shown as (4) in scheme 3 below (Hamilton et al, J. Am. Chem. Soc., 125:15049-15058 (2003); Joseph et al., J. Med. Chem., 46:194-196 (2003)). 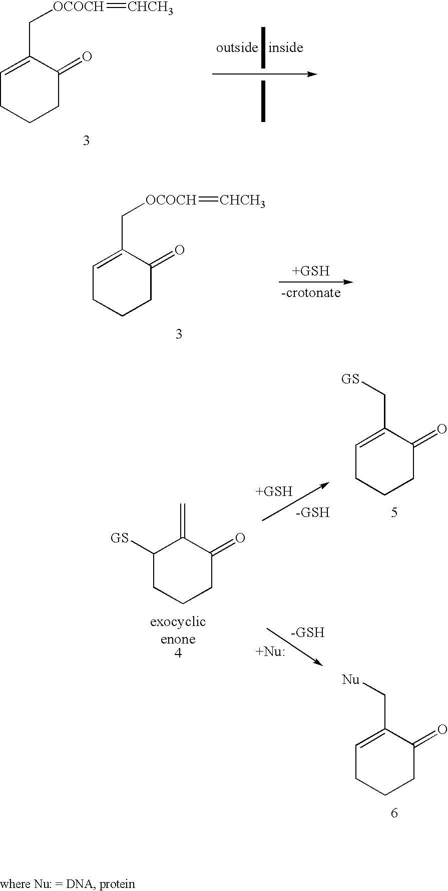
While not a GlxI inhibitor, this species can potentially react with either free GSH to give (5) or form covalent adducts with proteins and/or nucleic acids, shown as (6), critical to cell viability.

Indeed, mass spectral studies show that in the presence of GSH, 2-crotonyloxymethyl-2-cyclohexenone (shown as (3) in Scheme 3 above) alkylates the exocyclic amino groups of nucleotide bases composing single-stranded oligonucleotides (Zhang et al., Organic Lett., 4:1459-1462 (2002)).

However, neither of the prodrug strategies described above is designed to deliver antitumor agents specifically to tumor tissue. For example, in vivo efficacy studies show that intravenous administration of the diethyl ester of S—(N-4-chlorophenyl-N-hydroxycarbamoyl)glutathione, CHG(Et)₂, to tumor-bearing mice inhibits the growth of B16 melanotic melanoma, PC3 prostate tumors and HT29 human colon tumors (Sharkey et al, Cancer Chemotherapy and Pharmacology, 46:156-166 (2000)). While these short term efficacy studies did not detect any significant side effects, intravenous administration of CHG(Et)₂ results in the appearance of the prodrug in all major organs of tumor-bearing mice. This could give rise to significant side effects during long-term administration of the drug.

High molecular weight copolymer-prodrugs have been used to better direct cancer chemotherapeutic agents to tumor tissue via the so-called “enhanced permeability and retention” (EPR) effect (Brocchini and Duncan, “Pendent drugs, release from polymers,” In Encyclopedia of Controlled Drug Delivery, Mathiowitz Ed., pp 786-816, John Wiley and Sons, New York (1999); Duncan, Nat. Rev. Drug Discoc., 2:347-360 (2003); Thanou and Duncan, Curr. Opin. Investig. Drugs, 4:701-709 (2003); Duncan, Anti-Cancer Drugs, 3:175-210 (1992)).

This effect arises, in part, from the tendency of high molecular weight species to remain in circulating plasma longer than low molecular weight drugs, and from the fact that blood vessels in rapidly growing tumors are more permeable to high molecular weight species than are blood vessels in normal, established tissues. Once concentrated in tumor tissue, the high molecular weight prodrugs can then enter tumor cells by endocytosis.

The challenge is to design macromolecular prodrugs that will release the antitumor agents into the cell cytosol subsequent to endocytosis.

A typical strategy for designing copolymer prodrugs is to covalently attach the drug to the polymer via a peptide linkage, which will undergo catalyzed hydrolysis when the copolymer-prodrug-filled endosomes fuse with the peptidase-filled lysosomes. The free drug is then available to diffuse out of the lysosomes into the cell cytosol. Indeed, prodrug conjugates of polyhydroxypropylmethacrylamide (HPMA) have been designed to deliver the anticancer drugs doxorubicin (Seymour et al, Br. J. Cancer, 63:859-866 (1991)), daunomycin (Duncan et al, Br. J. Cancer, 57:147-156 (1988)), 5-fluorouracil (Putnam and Kopecek, Bioconjugate Chem., 6:483-492 (1995)), and the DNA alkylating agent melphalan (Duncan et al, J. Controlled Release, 16:121-136 (1991)) into tumor cells. Moreover, some of these conjugates are now entering Phase I/II clinical trials (Duncan, “Polymer-drug conjugates,” In Handbook of Anticancer Drug Development, Budman, Calvert, and Rowinsky Eds., pp 230-260, Lippinott, Williams and Wilkins, Baltimore (2003)).

A limitation of this approach is that cleavage of the peptide bond in the linker can be a slow process taking several hours (Seymour et al, Br. J. Cancer, 63:859-866 (1991)).

An alternative strategy was employed for antisense oligonucleotides that avoided reliance on lysosomal peptidases. In this strategy, the polymer and oligonucleotide were conjugated through a disulfide bond, to allow intracellular thiols and/or redox enzymes to release the oligonucleotides intracellularly (Wang et al., Bioconjugate Chem., 9:749-757 (1998)).

Such GSH-activated prodrugs are intriguing for cancer therapy because GSH concentrations are often elevated by as much as two-fold in tumor tissues (Cook et al, Cancer Res., 51:4287-4294 (1991); Blair et al, Cancer Res., 57:152-155 (1997); Kosower and Kosower, Int. Rev. Cyt., 54:109-160 (1978))) versus normal tissues, which are in the range 2-8 mM (Kosower and Kosower, Supra (1978); Meister, “Metabolism and transport of glutathione and other γ-glutamyl compounds,” In Functions of Glutathione: Biochemical, Physiological, Toxicological, and Clinical Aspects, Larsson, et al. Eds., pp 1-22, Raven Press, New York (1983)). Moreover, it has been reported that some drug resistant tumors have 10-fold higher levels of GSH (Britten et al., Int. J. Radiat. Oncol. Bio. Phys., 24:527-531 (1992)). Thus, these GSH concentration differences could give rise to preferential activation of a strategically designed GSH-activated macromolecular prodrug in tumor cells.

Further, the adventitious activation of a GSH-activated macromolecular prodrug in circulating human plasma should be minimal, as GSH concentrations are typically 1-2 μM (Anderson, “Enzymatic and Chemical Methods for Determination of Glutathione,” In Glutathione: Chemical, Biochemical, and Medical Aspects, Vol IIIA, Dolphin et al, Eds., John Wiley and Sons, Toronto (1989)).

While in theory macromolecular carriers could be conjugated to the sulfoxide prodrugs described above (see Scheme 2), for example as HPMA copolymers, to target the sulfoxide prodrugs preferentially to tumor tissue and to release active GlxI inhibitors preferentially in tumor cells, intracellular activation of such prodrugs would require sufficient levels of endosomal or lysosomal GSH. Further, it is unknown if GSH concentrations in endosomes or lysosomes are sufficient for release of active drug from the carrier. While studies have employed thiol-activated HPMA copolymers (Wang, et al (1998) supra), activation of these compounds did not specifically rely on lysosomal GSH.

While there are no good estimates of the steady-state concentrations of specific thiols inside the lysosomes of mammalian cells, there is some evidence to indicate that lysosomes contain transporter systems for importing cysteine and cysteinyl dipeptides like cysteinyl-glycine into lysosomes (Foster and Lloyd, Biochim. Biophys. Acta 947:465-491 (1988)).

For example, a highly specific cysteine-dependent transporter has been found in human fibroblast lysosomes with a Kₘ of 0.5 mM, and evidence has been presented for the efflux of cystine and cysteine from these lysosomes (Pisoni et al, J Cell Biol. 110:327-335 (1990)). An important role of cysteine is to activate intralysosomal thiol-dependent proteases that function to breakdown proteins taken up by endocytosis.

Early evidence that GSH also stimulates intralysosomal protein breakdown was attributed to a GSH transporter in the lysosomal membrane (Mego, Biochem J. 218:775-783 (1984)). However, subsequent studies indicate that some or perhaps all of the activation was due to breakdown of GSH to cysteinylglycine, which is subsequently transported into the lysosomes (Mego, Biochim. Biophys. Acta 841:139-144 (1985)).

Thus, prior to the present invention, the ultimate success of a macromolecular GSH-activated prodrug strategy could not be predicted.

### SUMMARY OF THE INVENTION

An object of the present invention is to provide macromolecular prodrugs of GSH-based antitumor agents that will accumulate preferentially in tumor tissue, and will be activated preferentially in tumor cells.

In one embodiment, this object has been met by covalently linking sulfoxide prodrugs to macromolecular carriers, which upon administration to a cancer patient, will help target the prodrug to tumor tissue via the “enhanced permeability and retention effect.” These sulfoxide prodrugs, which will be taken up largely via endocytosis by tumor cells, contain a sulfoxide group adjacent to an acyl group which undergoes an acyl interchange reaction with glutathione present in the enodosomal or lysosomal compartment. This acyl interchange reaction results in the release of the prodrug from the carrier and the simultaneous activation of the prodrug to an active GlxI inhibitor, which may then diffuse into the cytoplasm of the tumor cell to target the GlxI enzyme.

Another object of the present invention is to provide an effective antitumor pharmaceutical composition with less adverse side-effects than current chemotherapies.

According to one embodiment of the invention, this object has been met by macromolecular GSH-activated prodrugs in combination with a pharmaceutically acceptable diluent. Upon administration to a cancer patient, these macromolecular GSH-activated prodrugs accumulate preferentially in tumor tissue via the “enhanced permeability and retention effect.” Further, these macromolecular GSH-activated prodrugs may be activated preferentially in tumor cells having elevated concentrations of GSH.

A further object of the present invention is to provide a method of treating a subject having a neoplastic condition.

According to one embodiment of the invention, this object has been met by a method comprising the step of administering to a subject having a neoplastic condition a pharmaceutically effective amount of a macromolecular GSH-activated prodrug. As described above, these prodrugs accumulate preferentially in tumor tissue.

A still further object of the present invention is to provide a method of inhibiting the proliferation of a tumor cell.

According to one embodiment of the invention, this object has been met by a method comprising the step of contacting a tumor cell with an amount of a macromolecular GSH-activated prodrug that effectively inhibits proliferation of said tumor cell. The macromolecular prodrug is activated by endosomal or lysosomal GSH, after endocytotic uptake, allowing the active GlxI inhibitor to diffuse into the cytoplasm and exhibit antitumor activity.

Another object of the invention is to provide a method of forming an active GlxI inhibitor from a macromolecular prodrug in the presence of glutathione.

According to one embodiment of the invention, this object has been met by providing a sulfoxide prodrug covalently linked to a macromolecular carrier, such that, in the presence of glutathione, an active GlxI inhibitor will be formed and released from said carrier.

An additional object of the invention is to provide methods of synthesizing macromolecular copolymer prodrugs.

According to one embodiment of the invention, this object has been met by a method comprising the following steps. Performing an acyl interchange reaction with S—(N-aryl/alkyl-N-hydroxycarbamoyl)alkylsulfoxide and a thioamine to form S—(N-aryl/alkyl-N-hydroxycarbamoyl)thioalkylamine. Reacting S—(N-aryl/alkyl-N-hydroxycarbamoyl)thioalkylamine with methacryloyl chloride and pyridine so as to form S—(N-aryl/alkyl-N-hydroxycarbamoylthioalkyl)methacrylamide. Then, co-polymerizing S—(N-aryl/alkyl-N-hydroxycarbamoylthioalkyl)methacrylamide with an acrylamide to form a copolymer prodrug.

According to another embodiment of this invention, this object has been met by reacting 2-hydroxymethyl-2-endocyclic enone and methacryloyl chloride to form 2-methacyloyloxymethyl-2-endocyclic enone, and co-polymerizing the 2-methacyloyloxymethyl-2-endocyclic enone with an acrylamide to form the copolymer prodrug.

Other and further aspects, features, and advantages of the present invention will become apparent to a skilled artisan in view of the present disclosure of the invention as set forth herein.

### BRIEF DESCRIPTION OF THE DRAWINGS

FIG. 1 Mechanisms by which a GlxI inhibitor and an alkylating exocyclic enone could be generated from a copolymer-prodrug conjugate in the presence of GSH.

FIG. 2 Synthesis of HPMA polyacrylamide conjugates.

FIG. 3 Spectral data of an HPMA-copolymer containing both the sulfoxide and 2-methylenecyclohexenone functions: (top) IR (ATR, AMTIR); (bottom) ¹H NMR 300 MHz (methanol-d₄/TMS) spectra. Spectral lines unique to the three functional groups in the copolymers are indicated in the spectra.

FIG. 4 Mol compositions and spectrophotometrically determined second-order rate constants (k₂) for the reaction of GSH with the sulfoxide and cyclohexenone functions of different copolymer prodrugs. Kinetic constants were calculated from the first-order rate of loss of the functional group under pseudo-first-order conditions (>20-fold excess of GSH), and are the average of triplicate determinations ±standard deviation.

FIG. 5 Elution profile from a reverse-phase HPLC column of a reaction mixture obtained after incubating P8 with 10 mM GSH in potassium phosphate buffer (0.1 M), pH 6.5, for 15 min. Abscissa: percent total absorbancy (200-400 nm). Peaks at 1.02, 1.42 and 3.72 min correspond to GSH, the GSH-2-methylcyclohexenone adduct (designated as (5) in FIG. 1) and the transition state analogue (designated as (1) in FIG. 1), respectively).

FIG. 6 Time-dependent change in the UV spectrum of P3 (FIG. 6A) (0.025 mM in 8-sulfoxide) in potassium phosphate buffer (0.1 M, pH 6.5), GSH (0.5 mM), EDTA (0.05 mM), 2.5 vol % ethanol, 25° C. (spectral scans taken every 25 s) and of P6 (FIG. 6B) (0.05 mM in 7) in potassium phosphate buffer (0.1 M, pH 6.5), GSH (1.0 mM), 5 vol % ethanol, 25° C. (spectral scans taken every 10 min).

FIG. 7 Rate profiles for the reaction of copolymer P6 (FIG. 7A) (0.05 mM in cyclohexenyl equivalents) with GSH (1 mM), and copolymer P3 (FIG. 7B) (0.025 mM in 8-sulfoxide equivalents) with GSH (0.5 mM). The rate constants are the best-fit values to the expression for a first-order exponential decay (solid line through the data).

FIG. 8. In vitro inhibition of B16 melanotic melanoma by different HPMA copolymer prodrug conjugates versus the unpolymerized prodrugs.

FIG. 9 Growth inhibition of B16 melanoma in the presence of P1 (FIG. 9A) or P4 (FIG. 9B). Data for copolymer drug conjugates are indicated by squares; data for polymer controls with no appended drug are indicated by triangles. Drug concentration is calculated, on the basis of equivalents of drug/L.

### DETAILED DESCRIPTION OF THE INVENTION

The present invention provides prodrugs useful as anti-tumor agents that comprise a macromolecular carrier and at least one precursor of a GlxI inhibitor covalently linked to the macromolecular carrier. The precursor contains a sulfoxide adjacent to an acyl group which, in the presence of glutathione, allows the formation and release of an active GlxI inhibitor from the macromolecular carrier as a result of an acyl interchange reaction with the thiol of a glutathione at the acyl group of the precursor.

Due to the macromolecular carrier, the prodrug accumulates preferentially in tumor tissue via the enhanced permeability and retention effect, which is due in part to the fact that higher molecular weight species tend to remain in circulating plasma longer than low molecular weight species, and the fact that blood vessels in rapidly growing tumors are more permeable to high molecular weight species than are blood vessels in normal, established tissues.

Further, after endocytosis of the macromolecular prodrug by a tumor cell, endosomal or lysosomal glutathione will initiate release and activation of the active drug, which may then diffuse into the cytoplasm of the cell to target the GlxI enzyme. Because some tumor cells may have elevated levels of GSH, activation and release of the drug may also occur preferentially in tumor cells.

The efficacy of the invention described herein for the first time demonstrates that the concentration of GSH inside lysosomes and/or endosomes is sufficient to form active GlxI inhibitor from the corresponding sulfoxide prodrug (Scheme 2), as well as cytotoxic exocyclic enone from the corresponding endocyclic enone (Scheme 3).

The macromolecular carrier employed with the invention has an average molecular mass of greater than 2.5 kDa. Preferably, the macromolecular carrier has an average molecular mass within the range of 5 kDa to 70 kDa, more preferably in the range of 10 kDa to 50 kDa, and most preferably in the range of 15 kDa to 32 kDa.

Generally, larger molecular weight species will accumulate in tumor tissue via the EPR effect (Brocchini and Duncan, supra (1999)). Macromolecular prodrugs with a molecular weight of 20 kDa or greater may penetrate selectively through the leaky blood vessels in tumor tissue. Further, macromolecular prodrugs with a molecular weight of about 10 kDa or greater may avoid loss through the renal tubules.

Macromolecular carriers of the invention include carriers that are linear or branched polymers.

In one embodiment, the macromolecular carrier is polymerized from polymeric units having an amide. For example, the prodrug may be formed by radical polymerization of the polymeric amide unit and the methacrylamide derivative(s) of sulfoxide prodrugs and/or endocyclic enones. Preferred carriers in this regard are polyacrylamide and polymethacrylamide. Particularly preferred are N-(2-hydroxypropyl)methacrylamide (HPMA) as HPMA exhibits little immunogenicity (Rihova et al., Makromol. Chem. 9:13-24 (1985). HPMA is commercially available from Polysciences, Inc (Warrington, Pa.). Further, various derivatives, of acrylamide may be used in accordance with the invention which are known in the art and which are commercially available.

Still other types of macromolecular carriers that may be employed with the invention, and which are well-known in the art, include: polyethylene glycol (PEG); DIVEMA (Brocchini and Duncan, supra (1999)); polysaccharides such as dextran, chitosan, carboxymethylchitin, carboxymethylpullulan, and alginate; polyaminoacids; polyesters; block copolymers; and alternating polymers such as PEG lysine.

The precursors that may be covalently linked to macromolecular carriers in accordance with the invention are those that contain a sulfoxide adjacent to an acyl group, such that an acyl interchange reaction with the thiol of a glutathione will simultaneously form and release an active GlxI inhibitor from the carrier.

Preferred precursors contain an N-hydroxycarbamoyl moiety, which binds tightly to the human GlxI enzyme active site.

Particularly preferred are precursors defined by the formula S—(N-aryl/alkyl-N-hydroxycarbamoyl)alkyl sulfoxide. The synthesis of these ethyl sulfoxide prodrugs is known in the art (Hamilton et al, J. Med. Chem. 42:1823-1827).

The alkyl sulfoxide group may be a C₁-C₂₀ alkyl sulfoxide, preferably a C₁-C₁₀ alkyl sulfoxide. Of these, ethyl, propyl, butyl, pentyl, or hexyl sulfoxides are particularly preferred, with ethyl sulfoxide precursors being the most preferred.

The precursors may be S—(N-aryl-N-hydroxycarbamoyl)alkyl sulfoxides, where aryl is a carbocyclic or heterocyclic group, which may be substituted or unsubstituted, with at least one ring having a conjugated n-electron system, and containing up to two conjugated or fused ring systems. Heterocyclic aryls may contain C, N, O, or S atoms. Carbocyclic aryls are preferred, with substituted or unsubstituted phenyl being particularly preferred.

The precursors may be S—(N-alkyl-N-hydroxycarbamoyl)alkyl sulfoxides, where N-alkyl is a C₁-C₂₀ alkyl, and preferably a C₁-C₁₀ alkyl. Of these, ethyl, propyl, butyl, pentyl and hexyl are particularly preferred.

Particularly preferred precursors include: S—(N-p-chlorophenyl-N-hydroxycarbamoyl)ethyl sulfoxide, S—(N-4-chlorophenyl-N-hydroxycarbamoyl)propyl sulfoxide, S—(N-4-chlorophenyl-N-hydroxycarbamoyl)butyl sulfoxide, S—(N-p-bromophenyl-N-hydroxycarbamoyl)ethyl sulfoxide, S—(N-p-iodophenyl-N-hydroxycarbamoyl)ethyl sulfoxide, S—(N-phenyl-N-hydroxycarbamoyl)ethyl sulfoxide, S—(N-hydroxy-N-methylcarbamoyl)ethyl sulfoxide, S—(N-methyl-N-hydroxycarbamoyl)ethyl sulfoxide, S—(N-ethyl-N-hydroxycarbamoyl)ethyl sulfoxide, S—(N-propyl-N-hydroxycarbamoyl)ethyl sulfoxide, S—(N-butyl-N-hydroxycarbamoyl)ethyl sulfoxide, S—(N-pentyl-N-hydroxycarbamoyl)ethyl sulfoxide, and S—(N-hexyl-N-hydroxycarbamoyl)ethyl sulfoxide.

The precursors of the invention give rise to active GlxI inhibitors via an acyl interchange reaction with glutathione. Preferred precursors form an active GlxI inhibitor of the formula S—(N-aryl/alkyl-N-hydroxycarbamoyl)glutathione, which are transition state analogues of the GlxI enzyme. In these transition state analogues, aryl is a carbocyclic or heterocyclic group, which may be substituted or unsubstituted, with at least one ring having a conjugated n-electron system, and containing up to two conjugated or fused ring systems. Heterocyclic aryls may contain C, N, O, or S atoms. Carbocyclic aryls are preferred, with substituted or unsubstituted phenyl being particularly preferred.

Further, in these preferred active inhibitors, N-alkyl is a C₁-C₂₀ alkyl, and preferably a C₁-C₁₀ alkyl. Of these, ethyl, propyl, butyl, pentyl and hexyl are particularly preferred.

Particularly preferred active GlxI inhibitors of the formula S—(N-aryl-N-hydroxycarbamoyl)glutathione include: S—(N-p-chlorophenyl-N-hydroxycarbamoyl)glutathione, S—(N-p-bromophenyl-N-hydroxycarbamoyl)glutathione, S—(N-p-iodophenyl-N-hydroxycarbamoyl)glutathione, S—(N-phenyl-N-hydroxycarbamoyl)glutathione.

U.S. Pat. No. 5,616,563, which is hereby incorporated by reference, describes methods of synthesis for S—(N-hydroxycarbamoyl)glutathione derivatives including: S—(N-p-chlorophenyl-N-hydroxycarbamoyl)glutathione, S—(N-p-bromophenyl-N-hydroxycarbamoyl)glutathione, S—(N-p-iodophenyl-N-hydroxycarbamoyl)glutathione, S—(N-phenyl-N-hydroxy-carbamoyl)glutathione. Moreover, the synthesis of the S—(N-aryl/alkyl-N-hydroxycarbamoyl)glutathiones is described by Kalsi, et al, J. Med. Chem. 43, 3981-3986 (2000).

Other GlxI inhibitors that may be used in accordance with the invention include irreversible inactivators, which acylate the active site of the human glyoxylase I enzyme. These compounds, including the synthesis thereof, have been described in WO 2005/007079, the disclosure of which is hereby incorporated by reference.

Irreversible inactivators include those of the formula S—(CH₂C(O)OROC(O)CH₂X)glutathione, where R is selected from the group consisting of alkylene, (CH₂CH₂O)₁₋₂₀, (CH₂CH₂N)₁₋₂₀, and arylene. X represents a halogen.

Preferred irreversible inactivators are compounds of the formula CH₂C(O)O(CH₂)ₙOC(O)CH₂X) glutathione, wherein n is 2 through 6 and wherein X represents a halogen. Particularly preferred irreversible inactivators are S-(bromoacetoxy butyl acetoxy)glutathione and S-(bromoacetoxy propyl acetoxy)glutathione.

Computational docking of these compounds into the X-ray crystal structure of hGlxI indicates that the S-substituents are ideally positioned to alkylate the sulfhydryl group of Cys60 in the active site, which is located about 12 to 13 Angstroms from the sulfur atom of the bound inactivators. Other irreversible inactivators are also accommodated by the hGlxI active site, especially where the S-substituent is able to assume a “bowed” conformation in the active site, allowing the haloacetyl function to be positioned near Cys60.

Preferred irreversible inactivators have bromoacetoxy, chloroacetoxy, acryloyl and crotonyl groups. Particularly preferred are irreversible inactivators having a bromoacetoxy group.

Thus, these irreversible inactivators may be employed as sulfoxide prodrugs, analogous to the transition state analogues described supra, such that the inactivator is formed and released from a macromolecular carrier through an acyl interchange reaction with GSH.

While any GlxI inhibitor which may be formed by an acyl interchange reaction between a sulfoxide precursor and glutathione may be employed according to the invention, one skilled in the art understands is that the hydrophobicity of the resulting S-substituent of the active drug correlates with a higher affinity for the human GlxI active site (Kalsi 2000, supra). The S—(N-aryl-N-hydroxycarbamoyl)glutathione derivatives bind especially tightly to the active site of GlxI, as these compounds mimic the stereoelectronic features of the tightly bound transition state formed along the reaction coordinate of the enzyme during normal catalysis.

Other preferred embodiments of the invention employ human GlxI inhibitor(s) that are catalytically hydrolyzed by human GlxII, a thioester hydrolase that is abundant in normal tissues, but deficient in tumor tissues. This is an additional basis for tumor targeting, since such compounds are believed to accumulate specifically in tumor cells. For example, S—(N-p-chlorophenyl-N-hydroxycarbamoyl)glutathione, S—(N-p-bromophenyl-N-hydroxycarbamoyl)glutathione, S—(N-phenyl-N-hydroxycarbamoyl)glutathione, and S—(N-methyl-N-hydroxycarbamoyl)glutathione are substrates for bovine liver GlxII.

The prodrugs of the invention may comprise two or more precursors which may be the same or different. Further, the prodrugs may also comprise one or more alkylating agents, such as an endocyclic enone that gives rise to the exocyclic enone through a Michael addition reaction with glutathione. These compounds will also be released from the carrier as a result of the Michael addition reaction. As with the GlxI inhibitors, these compounds may be activated preferentially in tumor cells having elevated concentrations of glutathione. Preferably, the macromolecular prodrug is in the form of an HPMA copolymer with one or more sulfoxide precursors and one or more endocyclic enones.

These alkylating agents, including the synthesis thereof, have been described in U.S. application Ser. No. 10/098,834, published as 2003/0191066, which is incorporated herein by reference in its entirety.

Preferred endocyclic enones that may act as precursors to alkylating agents are 2-substituted cyclohexenone, 2-substituted cycloheptenone, 2-substituted cyclopentenone, 2-substituted benzoquinone, 2-substituted napthoquinone, and 2-substituted anthroquinone.

Other alkylating agents include the COMC derivatives COMC-5, COMC-6, COMC-7, and COMC-8 (Hamilton et al., J. Am. Chem. Soc. 25:15049 (2003)).

Preferred embodiments of the invention include prodrugs comprising copolymers of HPMA along with one or more of the following precursors of transition state analogues: S—(N-p-chlorophenyl-N-hydroxycarbamoyl)ethyl sulfoxide, S—(N-4-chlorophenyl-N-hydroxycarbamoyl)propyl sulfoxide, S—(N-4-chlorophenyl-N-hydroxycarbamoyl)butyl sulfoxide, S—(N-p-bromophenyl-N-hydroxycarbamoyl)ethyl sulfoxide, S—(N-p-iodophenyl-N-hydroxycarbamoyl)ethyl sulfoxide, S—(N-phenyl-N-hydroxycarbamoyl)ethyl sulfoxide, S—(N-hydroxy-N-methylcarbamoyl)ethyl sulfoxide, S—(N-methyl-N-hydroxycarbamoyl)ethyl sulfoxide, S—(N-ethyl-N-hydroxycarbamoyl)ethyl sulfoxide, S—(N-propyl-N-hydroxycarbamoyl)ethyl sulfoxide, S—(N-butyl-N-hydroxycarbamoyl)ethyl sulfoxide, S—(N-pentyl-N-hydroxycarbamoyl)ethyl sulfoxide, and S—(N-hexyl-N-hydroxycarbamoyl)ethyl sulfoxide; and one or more of the following precursors of alkylating agents: 2-substituted-2-cyclohexenone, 2-substituted-2-cycloheptenone, 2-substituted-2-cyclopentenone, 2-substituted benzoquinone, 2-substituted napthoquinone, and 2-substituted anthroquinone.

Preferably, the mol % of the sulfoxide precursor and/or endocyclic enone in the macromolecular prodrug of the invention is at least 1.5, more preferably at least 3, and still more preferably at least 8. Most preferably, the sulfoxide precursor and/or endocyclic enone in the macromolecular prodrug is about 10 mol %.

The present invention further provides pharmaceutical compositions comprising a macromolecular prodrug of the invention together with a pharmaceutically acceptable diluent, such as physiological saline. In some embodiments, the pharmaceutically acceptable diluent includes DMSO as needed to solubilize the prodrug.

The present invention further provides methods of treating a subject having a neoplastic condition comprising administering to a subject in need of such treatment a pharmaceutically effective amount of a macromolecular GSH-activated prodrug.

The particular amount administered varies depending on the age, weight, sex of the subject, the mode of administration, and the particular neoplastic condition being treated.

Typically, a pharmaceutically effective amount is a dose of from 0.01 g of macromolecular prodrug containing from 8 to 10 mol % inhibitor and/or alkylating agent to about 1.0 g of macromolecular prodrug. A preferred dose is from 0.1 g to about 1.0 g.

Further, a pharmaceutically effective dose may also be extrapolated from in vitro cytotoxic studies as well as from animal studies.

The compositions of the invention may be administered to treat a neoplastic condition. Generally, the compositions of the present invention can be used to treat any cancerous condition. Preferred conditions are members selected from the group consisting of breast cancer, ovarian cancer, prostate cancer, lung cancer, colon cancer, kidney cancer, liver cancer, brain cancer, and haemopoetic tissue cancer. More preferred cancers are prostate, colon and lung tumors, which overexpress GlxI, as these tumors have previously been shown to be particularly sensitive to GlxI inhibition by competitive GlxI inhibitors (Sharkey et al 2000, supra; Sakamoto et al (2001), supra).

Preferably, the tumor to be treated is particularly susceptible to the “enhanced permeability and retention effect,” such that the macromolecular prodrug accumulates in the tumor tissue selectively.

Although the compositions of the present invention may be administered in any favorable fashion, intravenous, subcutaneous, and intramuscular administration are preferred. Most preferably, the compositions of the invention are administered intravenously.

The composition of the present invention may be administered by continuous i.v. infusion or bolus i.v. infusion to a subject having a neoplastic condition. In vivo efficacy studies of the diethyl ester prodrugs in tumor-bearing mice suggest that slow growing tumors are preferably treated by continuous infusion, while rapidly growing tumors are preferably treated by i.v. bolus administration (Sharkey et al 2000, supra).

The present invention also provides methods of inhibiting the proliferation of a tumor cell comprising contacting a tumor cell with an amount of a composition of the invention effective to inhibit proliferation of said tumor cell. Thus, the present invention includes inhibiting the proliferation of a tumor cell in vitro as well as in vivo.

An effective amount of the macromolecular prodrug of the invention for inhibiting proliferation of a tumor cell in vivo is that which provides a concentration of drug that results in a 500 decrease in tumor volume over the course of treatment. A pharmaceutically effective amount may be from 0.01 g of macromolecular prodrug containing from 8 to 10 mol % inhibitor and/or alkylating agent to about 1.0 g of macromolecular prodrug. A preferred dose is from 0.1 g to about 1.0 g. Of course, the particular amount administered varies depending on the age, weight, sex of the subject, the mode of administration, and the particular neoplastic condition being treated.

An effective amount of a macromolecular prodrug of the invention effective to inhibit proliferation of a tumor cell in vitro is that which provides a concentration of drug in a range of about 50 nM to about 1 mM, and preferably 300 nM or less.

The invention provides methods of forming active GlxI inhibitor and/or alkylating exocyclic enone from a macromolecular prodrug. In one embodiment of the invention, active GlxI inhibitor and/or alkylating exocyclic enone is formed by contacting the prodrug with glutathione. The prodrug may be contacted with glutathione in vivo or in vitro. In one embodiment, the macromolecular prodrug of the invention is contacted in vitro with a 20-fold excess of glutathione

The present invention also provides methods of synthesizing copolymer prodrugs.

In one embodiment of the invention, the method comprises the following steps. Performing an acyl interchange reaction between S—(N-aryl/alkyl/hydroxy-N-hydroxycarbamoyl)alkyl sulfoxide and a thioamine to form S—(N-aryl/alkyl-N-hydroxycarbamoyl)thioalkylamine. S—(N-aryl/alkyl-N-hydroxycarbamoyl)thioalkylamine is then reacted with methacryloyl chloride and pyridine to form S—(N-aryl/alkyl-N-hydroxycarbamoylthioalkyl)methacrylamide, which is then polymerized with an acrylamide to form a copolymer prodrug.

In a preferred embodiment the thioamine is cysteamine. Further, in preferred embodiments S—(N-aryl/alkyl-N-hydroxycarbamoylthioalkyl)methacrylamide is copolymerized with HPMA in the presence of azobisisobutylnitrile in an organic solvent such as acetone to form the copolymer prodrug.

The invention also provides methods of synthesizing copolymer prodrugs which comprise endocyclic enones capable of forming the alkylating exocyclic enone upon reaction with GSH. According to the invention, copolymer prodrugs may be produced by reacting 2-hydroxymethyl-2-endocyclic enone and methacryloyl so as to form 2-methacryloyloxymethyl-2-endocyclic enone, and then co-polymerizing the 2-methacryloyloxymethyl-2-endocyclic enone with an acrylamide to form a copolymer prodrug.

In a preferred embodiment, 2-methacryloyloxymethyl-2-endocyclic enone is formed in a reaction with 4-methylmorpholine. Further, in preferred embodiments methacryloyloxymethyl-2-endocyclic enone is copolymerized with HPMA in the presence of azobisisobutylnitrile in an organic solvent such as acetone.

In particularly preferred embodiments, aryl/alkyl-N-hydroxycarbamoylthioalkyl)methacrylamide is copolymerized with HPMA and methacryloyloxymethyl-2-endocyclic enone in the presence of azobisisobutylnitrile in an organic solvent such as acetone.

The following examples are given for the purpose of illustrating various embodiments of the invention and are not meant to limit the present invention in any fashion.

### EXAMPLES

Analytical instrumentation. NMR spectra were taken on a GE QE-300 NMR spectrometer. IR spectra measured with a ThermoNicolet Avatar 370 FTIR spectrometer using a Pike MIRacle ATR accessory (AMTIR crystal). Mass spectral data were obtained at the Center for Biomedical and Bio-organic Mass Spectrometry, Washington University. UV spectra were recorded using a Beckman DU 640 spectrophotometer. HPLC was carried out using a Waters High-Performance Liquid Chromatography System composed of a 600 Controller, Delta 600 Pumps and 996 Photodiode Array Detector. Analytical HPLC was performed using a Waters Nova-Pak C₁₈, 4 μm, 3.9×150 mm column or Symmetry C₁₈, 5 μm, 4.6×150 mm column. Preparative HPLC was performed using a SymmetryPrep C₁₈ 7 μm, 19×150 mm column. The molecular weights of the HPMA copolymers were estimated by gel permeation chromatograph (GPC) using a High Performance Gel Permeation Column (Tricorn Superose 12 10/300 GL) from Amersham Biosciences.

Materials. Dextran molecular weight standards were purchased from Sigma Chem. Co. (1,000, 5,000, and 12,000 Da) and Polysciences, Inc. (40,000 Da). Lyophilized human serum and GSH were purchased from Sigma Chem. Co. HPMA was purchased from Polysciences, Inc. Azobisisobutylnitrile (AIBN), methacryloyl chloride, 4-methylmorpholine and 3-chloroperoxybenzoic acid (80-85% pure) were purchased from Aldrich Chem. Co. All other reagents were of the highest purity commercially available.

### Example 1

### Synthesis of Compounds and HPMA Copolymers

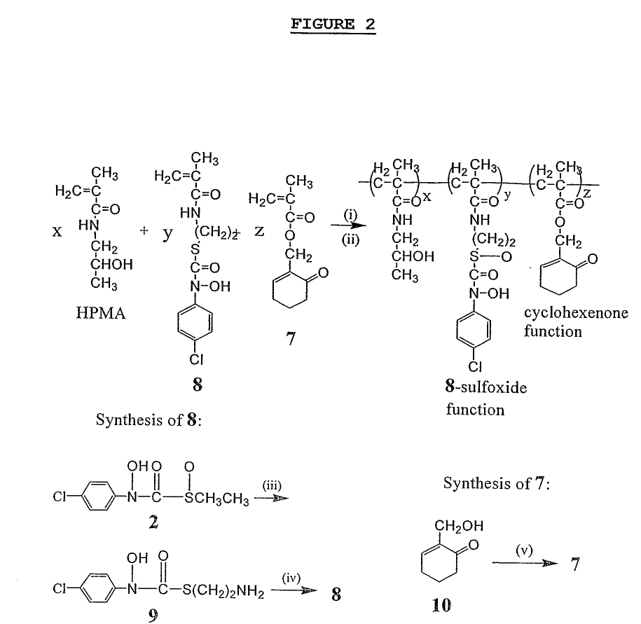

S—(N-4-Chlorophenyl-N-hydroxycarbamoyl)thioethylamine (shown as (9) in FIG. 2) was synthesized from the corresponding ethyl sulfoxide prodrug using the following procedure.

To a solution of S—(N-4-chlorophenyl-N-hydroxycarbamoyl)ethyl sulfoxide (shown as (2) in FIG. 2) (495 mg, 2 mmol) in a mixture of 12 mL methanol and 12 mL phosphate buffer (0.1 M, pH 7.5) was added a solution of cysteamine (990 mg, 12.9 mmol) in 5 mL of phosphate buffer (0.1 M, pH 7.5). The mixture was stirring at 0° C. for 1 h. The precipitate was collected by filtration, washed with water and dried under vacuum to give the final product as a white solid: Yield 86′ (424 mg). ¹H NMR (300 MHz, methanol-d₄/TMS) δ 3.01 (2H, t, J=6.6 Hz), 3.50 (2H, t, J=6.6 Hz) 7.32 (2H, d, J=9.2 Hz), 7.61 (2H, d, J=9.2 Hz); HRMS (ESI) m/z 247.0294 (calc'd for C₉H₁₂N₂O₂SCl: 247.0308). This procedure must form the thiol ester and not the amide because the “sulfoxide” containing copolymers react with free GSH to give the enediol analogue at rates similar to that observed for the reaction of GSH with the simple sulfoxide to give 1 (scheme 2).

S—(N-4-Chlorophenyl-N-hydroxycarbamoylthioethyl)methacrylamide (shown as (8) in FIG. 2) was synthesized from S—(N-4-Chlorophenyl-N-hydroxycarbamoyl)thioethylamine using the following procedure.

Methacryloyl chloride (318 μL, 3.29 mmol) was added slowly over 30 minutes to a stirring solution of S—(N-4-Chlorophenyl-N-hydroxycarbamoyl)thioethylamine (410 mg, 1.66 mmol) in 26 mL anhydrous DMF and 13 mL pyridine at 0° C., and the reaction mixture stirred at room temperature for an additional 20 minutes. The solvent was removed in vacuo and the residue fractionated by reverse-phase HPLC, using 50% acetonitrile in water, containing 0.1% trifluoroacetic acid, as a running solvent. The product peak was collected and dried under vacuum overnight to give the final product as a white solid: Yield 51% (269 mg). ¹H NMR (300 MHz, methanol-d₄/TMS) δ 1.90 (3H, s), 3.00 (2H, t, J=6.6 Hz), 3.45 (2H, t, J=6.6 Hz), 5.34 (1H, br s), 5.67 (1H, br s), 7.32 (2H, d, J=9.2 Hz), 7.59 (2H, d, J=9.2 Hz); HRMS (ESI) m/z 337.0379 (calc'd for [M+Na]⁺, C₁₃H₁₅N₂O₃NaSCl: 337.0390).

2-Methacryloyloxymethyl-2-cyclohexenone (shown as (7) in FIG. 2) was synthesized in the following manner.

Methacryloyl chloride (0.79 ml, 8 mmol) was added dropwise over about 30 minutes to a stirring solution of 2-hydroxymethyl-2-cyclohexenone (shown as (10) in FIG. 2) (504 mg, 4 mmol) and 4-methylmorpholine (1 mL, 9 mmol) in 10 mL CH₂Cl₂ at 0° C. The reaction mixture was allowed to stir for an additional 20 minutes. The solvent was removed in vacuo and the crude product fractionated by preparative reverse-phase HPLC using 40% acetonitrile in water, containing 0.1% trifluoroacetic acid, as a running solvent. The product peak was collected, and brought to dryness under vacuum to give the final product as a colorless oil: Yield 660 (515 mg) ¹H NMR (300 MHz, acetone-d₆) δ 1.96 (3H, s), 2.03 (2H, p, J=6.2), 2.48 (2H, t, J=7.33 Hz), 2.42-2.60 (2H, m), 4.84 (2H, s), 5.58 (1H, br s), 6.13 (1H, br s), 7.01 (1H, t, J=4.3 Hz); HR-FABMS (3-NBA/Li) m/z 201.1100 (calc'd for [M+Li]⁺, C₁₁H₁₄O₃Li: 201.1103).

Copolymer P1. The methacryloyl derivative shown as (8) in FIG. 2 (41 mg, 0.13 mmol), HPMA (110 mg, 0.77 mmol) and AIBN (6 mg) were dissolved in 0.75 ml acetone under argon in a closed vial, and incubated at 50-55° C. for 24 hours. The white precipitate was recovered by filtration and was dried under vacuum for 30 min. The crude product was dissolved in 0.25 mL of methanol and then precipitated by the slow addition of excess acetone:diethyl ether (3:1). The precipitation procedure was repeated and the precipitate dried under vacuum over night: Yield 63 mg.

A portion of the residue (30 mg) was dissolved in 1 mL of methanol at 0° C., and 3-chloroperoxybenzoic acid (5.6 mg, 0.017 mmol) in 0.05 mL of diethyl ether was slowly added dropwise to this solution. The reaction mixture was allowed to stir for an additional 1 h at 0° C. The solvent was removed in vacuo, the residue was dissolved in 0.2 mL of methanol and the desired product precipitated by the slow addition of 5 ml excess diethyl ether. The precipitation procedure was repeated twice more and the product was dried under vacuum overnight to give the final product: Yield overall from (8) and HPMA, 12 mg. IR (ATR, AMTIR): br 3352, 2973, 2934, 2907, s1636, s1526, s1487 (S—O), 1202 cm⁻¹.

Copolymers P2, P3. These copolymers were prepared by the same general procedure used to prepare P1, with reaction mixtures having the following composition: P2, compound (8) of FIG. 2 (21 mg, 0.067 mmol), HPMA (76 mg, 0.53 mmol) and AIBN (2 mg) dissolved in 0.4 mL acetone. Neat acetone was used as a precipitant during the precipitation procedures. Mass yield 29%. IR (ATR, AMTIR): br 3349, 2972, 2930, 2886, s1636, s1527, 1487 (S—O), 1203 cm⁻¹. P3, compound (8) of FIG. 2 (42 mg, 0.13 mmol), HPMA (282 mg, 2 mmol) and AIBN (18 mg) dissolved in 1.6 mL acetone: Yield 32 mg. IR (ATR, AMTIR): br 3353, 2972, 2934, 2905, s1638, s1527, 1487 (S—O), 1202 cm⁻¹.

¹H NMR (300 MHz, methanol-d₄/TMS) spectra of P1-P3. The spectra were all very similar to one another, with significant line broadening due to the high molecular weights of the copolymers. The chemical shift assignments were based on comparisons with the NMR spectra of poly HPMA and compound (8) of FIG. 2; relative integrated intensities varied as a function of the mole % of the 8-sulfoxide function: δ 1.01 (s, CH₃C(C)₃), 1.16 (d, CH₃CO—), 1.6-2.0 (m, —(CH₂)—HPMA), 2.9-3.3 (m, —NCHₐH_{b}CO—; N—CH₂CH₂—S), 3.87 (m, —HCO—), 7.40 (d, arom. H, meta to Cl), 7.70 (d, arom. H, ortho to Cl). The mol % of the 8-sulfoxide function in the different polymers was estimated from the integrated intensities of the aromatic ring protons (δ7.40, 7.70) versus that of H—C—O (δ 10.02): P1, ˜10; P2, ˜9; P3, ˜5.

Copolymers P4-P6. These copolymers were prepared by the same general procedure used to prepare P1 with the exception that the oxidation step with 3-chloroperoxybenzoic acid was not used.

P4, compound (7) of FIG. 2 (68 mg, 0.351 mmol), HPMA (100 mg, 0.702 mmol) and AIBN (9 mg) dissolved in 0.8 mL acetone: Yield 38 mg. IR (ATR, AMTIR): br 3366, 2970, 2931, m 1723 (C═O, cyclohexenone function), s 1639, s 1527, 1175, 1138 cm⁻¹.

P5, compound (7) of FIG. 2 (44 mg, 0.227 mmol), HPMA (130 mg, 0.907 mmol) and AIBN (9 mg) dissolved in 0.7 mL acetone. Yield 112 mg. IR (ATR, AMTIR): br 3362, 2970, 2929, m1724 (C═O, cyclohexenone function), s 1639, s 1528, 1199, 1138 cm⁻¹.

P6, compound (7) of FIG. 2 (15 mg, 0.076 mmol), HPMA (87 mg, 0.61 mmol) and AIBN (5 mg) dissolved in 0.5 mL acetone. Yield 52 mg. IR (ATR, AMTIR): br 3354, 2971, 2931, m 1721 (C═O, cyclohexenone function), s1637, s 1527, 1201, 1137 cm⁻¹.

¹H NMR (300 MHz, methanol-d₄/TMS) spectra of P4-P6. The spectra were all very similar to one another, with significant line broadening due to the high molecular weights of the copolymers. The chemical shift assignments were based on comparisons with the NMR spectra of poly HPMA, and compounds (7) and (8) of FIG. 2; relative integrated intensities varied as a function of the mole % of the cyclohexenone function derived from (7): δ 1.01 (s, CH₃C(C)₃), 1.16 (d, CH₃CO—), 1.6-2.0 (m, —(CH₂)—HPMA), 2.0-2.15 (m, —(C(4)H₂)-cyclohex-2-enone ring), 2.45-2.60 (m, —(C(5)H₂, C(6)H₂)-cyclohex-2-enone ring), 2.9-3.3 (m, —NCHₐH_{b}CO—; N—CH₂CH₂—S), 3.87 (m, —HCO—), 7.2-7.3 (t, vinyl-H). The mol % of the cyclohexenone function was estimated from the integrated intensities of the cyclohexenone ring protons C(5)H₂, C(6)H₂ (δ9.85) versus that of H—C—O (δ10.02): P4, ˜27; P5, ˜20; P6, ˜12.

Copolymer P7. A solution of compound (7) of FIG. 2 (15 mg, 0.076 mmol), compound (8) of FIG. 2 (24 mg, 0.076 mmol), HPMA (87 mg, 0.61 mmol) and AIBN (6 mg) in 0.6 ml acetone under argon in a closed vial was heated at 50-55° C. for 24 h. The white precipitate was recovered by filtration and brought to dryness to give 70 mg crude product. The crude product was dissolved in 2 ml methanol at 0° C. and m-chloroperoxybenzoic acid (9.1 mg, 0.042 mmol) in 0.1 mL of diethyl ether was added dropwise. The reaction mixture was allowed to stir for an additional 1 h, the solvent was removed in vacuo and the residue was dissolved in 0.4 mL methanol. The product was precipitated by the dropwise addition of acetone:diethyl ether (3:1). The precipitation procedure was repeated, and the final copolymer product was dried under vacuum overnight: Yield 52 mg. IR (ATR, AMTIR)): br 3364, 2970, 2930, m 1721 (C═O, cyclohexenone function), 1638, 1527, s 1487 (S—O), 1200, 1137 cm⁻¹.

Copolymer P8. P8 was prepared by the same general procedure used to prepare P7 starting with a reaction mixture composed of compound (7) (16 mg, 0.084 mmol), compound (8) (26 mg, 0.084 mmol), HPMA (191 mg, 1.336 mmol) and AIBN (12 mg) dissolved in 1.2 mL acetone: Yield 91 mg. IR (ATR, AMTIR): br 3365, 2971, 2931, m 1723 (C═O, cyclohexenone function), 1639, 1527, s 1482 (S—O), 1201, 1138 cm⁻¹.

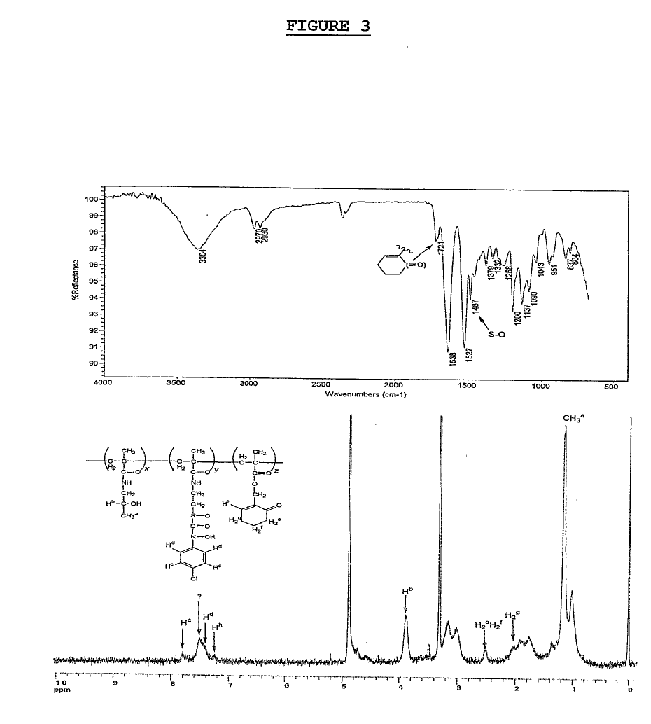

¹H NMR (300 MHz, methanol-d₄/TMS) spectra of P7 and P8. FIG. 3 shows that the spectra were both similar to one another, with significant line broadening due to the high molecular weights of the copolymers. The chemical shift assignments were based on comparisons with the NMR spectra of poly HPMA, compound (8) and (7); relative integrated intensities varied as a function of the mole % cyclohexenone and 8-sulfoxide functions: δ 1.01 (s, CH₃C(C)₃), 1.16 (d, CH₃CO—), 1.6-2.0 (m, —(CH₂)—HPMA), 2.0-2.15 (m, —(C(4)H₂)-cyclohex-2-enone ring), 2.45-2.60 (m, —(C(5)H₂, C(6)H₂)-cyclohex-2-enone ring), 2.9-3.3 (m, —NCHₐH_{b}CO—), 3.87 (m, —HCO—), 7.2-7.3 (t, vinyl-H), 7.40 (d, arom. Hs, meta to Cl), 7.70 (d, arom. Hs, ortho to Cl).

The mol % of the 8-sulfoxide and the cyclohexenone functions were estimated from the integrated intensities of the aromatic ring protons (δ7.40, 7.70) and cyclohexenone ring protons C(5)H₂, C(6)H₂ (δ 2.5) versus that of H—C—O (δ3.9): For P7, mol % 8-sulfoxide ˜2%, cyclohexenone function ˜2%; for P8, mol % 8-sulfoxide ˜6%, cyclohexenone function ˜6.

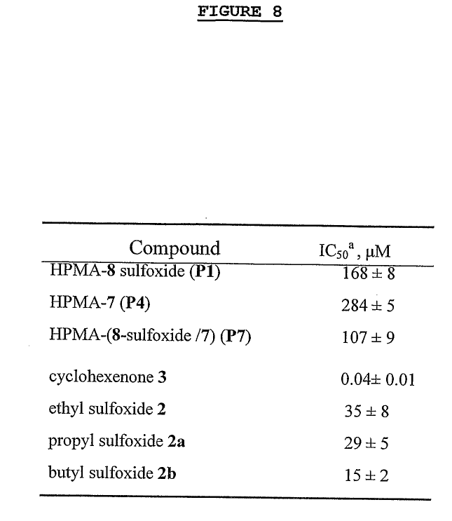

S—(N-4-Chlorophenyl-N-hydroxycarbamoyl)propyl sulfoxide (compound (2a) in FIG. 8). To S—(N-4-chlorophenyl-N-hydroxycarbamoyl)ethyl sulfoxide (compound (2) of FIG. 2) (150 mg, 0.61 mmol) in 3 mL pyridine on ice was added thiopropane (138 mg, 1.187 mmol). The reaction mixture was brought to room temperature, stirred for 30 minutes and aqueous 1.6 N HCl (15 mL) was slowly added to the stirring reaction mixture. The reaction mixture was extracted with methylene chloride (3×10 mL), the organic layer was dried over sodium sulfate and the solvent removed in vacuo. The residue was crystallized from hexane to give the synthetic intermediate S—(N-4-chlorophenyl-N-hydroxycarbamoyl)thiopropyl ester as light brown crystals (yield 41%). The ester (0.252 mmol) was dissolved in 2 mL diethyl ether to which was added dropwise 3-chloroperoxybenzoic acid (0.248 mmol) in 2 mL diethyl ether. The white precipitate was collected and washed with diethyl ether to give the final product: Yield from the intermediate ester was 48%. ¹H NMR (300 MHz, methanol-d₄/TMS) δ 0.99 (3H, t, J=7.3 Hz), 1.73 (2H, m), 3.04 (2H, m), 7.37 (2H, d, J=9.2 Hz), 7.68 (2H, d, J=9.2 Hz).

S—(N-4-Chlorophenyl-N-hydroxycarbamoyl)butyl sulfoxide (compound (2b) in FIG. 8). This compound was prepared by a procedure analogous to that used to prepare (2a) above: Yield 29%. ¹H NMR (300 MHz, CDCl₃/TMS) δ 0.87 (3H, t, J=7.3 Hz), 1.40 (2H, m), 1.70 (2H, m), 3.08 (2H, m), 7.37 (2H, d, J=8.8 Hz), 7.69 (2H, d, J=8.8 Hz).

### Characterization of the Copolymers

As described above, the copolymer prodrugs were prepared by radical polymerization of variable amounts of 2-methacryloylmethyl-2-cyclohexenone (compound (7) of FIG. 2) and/or N-(-4-chlorophenyl-N-hydroxycarbamoylthioethyl)methacrylamide (compound (8) of FIG. 2) with HPMA, followed by treatment with 3-chloroperoxybenzoic acid to oxidize the thioester function to a sulfoxide. Repeated precipitation from methanol/diethyl ether solution gave copolymer preparations that were free of unreacted 8-sulfoxide and/or compound (7) by HPLC. The yields were in the range 28-64%. The polyacrylamide monomers were prepared by an extension of published procedures (Hamilton et al, J. Med. Chem. 42:1823-1827 (1999); Hamilton et al., J. Amer. Chem. Soc. 125:15049-15058 (2003)).

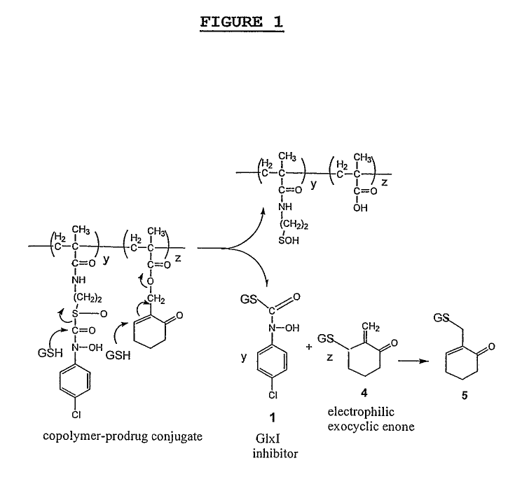

IR and NMR spectroscopy (FIG. 3) confirmed the chemical identities of the polymers. The 1487 and 1721 cm⁻¹ bands were assigned to the S—O and C═O stretching frequencies of the 8-sulfoxide and cyclohexenenone functions, respectively, on the basis of comparisons with the IR spectra of compounds (2) and (10). The integrated intensities of the resonances shown in the NMR spectrum could be used to calculate approximate values for the mol % compositions of the copolymers. However, more precise values were obtained by reacting the copolymers with GSH and quantitating the GSH adducts, compounds (1) and (5) of FIG. 1 after isolation by HPLC (described below).

The molecular weights of the copolymer prodrugs were determined as follows. The number average molecular weight (Mₙ), weight average molecular weight (M_{w}) and polydispersity (M_{w}/Mₙ) of the HPMA copolymers were obtained by high performance gel permeation chromatography (Strohalm et al, Angew. Makromol. Chem. 70:109-118 (1978)). The gel permeation column was eluted with 50 mM sodium phosphate buffer (pH 7.0) containing 0.25 M NaCl at a flow rate of one ml/min. Molecular weights were interpolated from standard curves (log Mₙ and log M_{w} versus retention time) obtained using polydextran molecular weight standards.

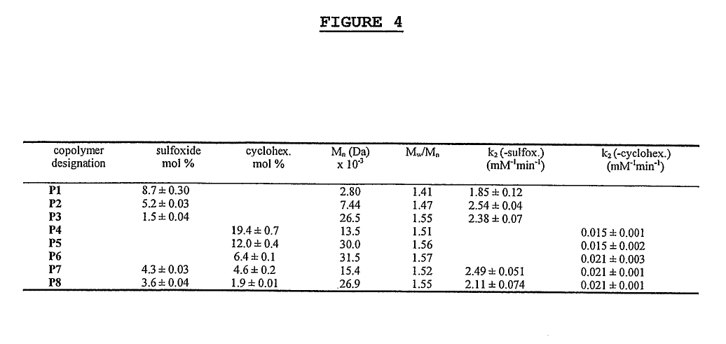

The results are shown in FIG. 4.

The mol % sulfoxide and cyclohexenone functions in the copolymers were determined as follows. Ethanolic solutions of copolymer were prepared and 20 μL aliquots were mixed with 80 μL of 10 mM GSH in potassium phosphate buffer (0.1 M, pH 7.5) and incubated at room temperature for 10 minutes to convert the 8-sulfoxide and/or cyclohexenyl functions to compounds (1) and the GSH adduct (5) of FIG. 1, respectively. A 10 μL aliquot of this solution was then fractionated by reverse-phase HPLC (Nova-Pak C₁₈, 4 μm, 3.9×150 mm column). For the analysis of compound (1), the running solvent was 25% acetonitrile in water, containing 0.1% trifluoroacetic acid; for 5, the running solvent was 13% acetonitrile in water, containing 0.1% trifluoroacetic acid. The areas under the peaks corresponding to compounds (1) and (5) were converted to mole quantities by comparison with standard curves of peak areas versus moles of authentic (1) and (5) injected onto the same column. The amounts of compounds (1) and (5) were then used to calculate mole fractions of 8-sulfoxide and cyclohexenyl groups in the original copolymer.

The results are shown in FIG. 4.

### Example 2

### Kinetics of Drug Release

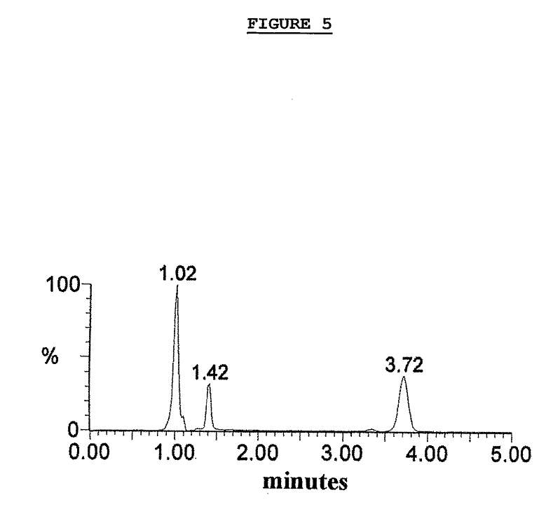

Reactions were initiated by the introduction of ethanolic solutions of copolymer into cuvettes containing at least a 20-fold excess of GSH over the equivalents of 8-sulfoxide and/or cyclohexenyl groups in degassed/N₂ saturated potassium phosphate buffer (0.1 M), pH 6.5, 25° C. Rate constants were calculated from the first-order decrease in absorbancy at 305 nm and 235 nm resulting from the loss of 8-sulfoxide and cyclohexenyl groups, respectively, FIG. 5.

Incubation of the HPMA copolymers with GSH produced a time-dependent increase in product species, which co-migrated with authentic samples of transition state analogue (i.e. compound (1)) and GSH-2-methylcyclohexenone adduct (i.e. compound (5)) monitored by reverse-phase HPLC; e.g., FIG. 5.

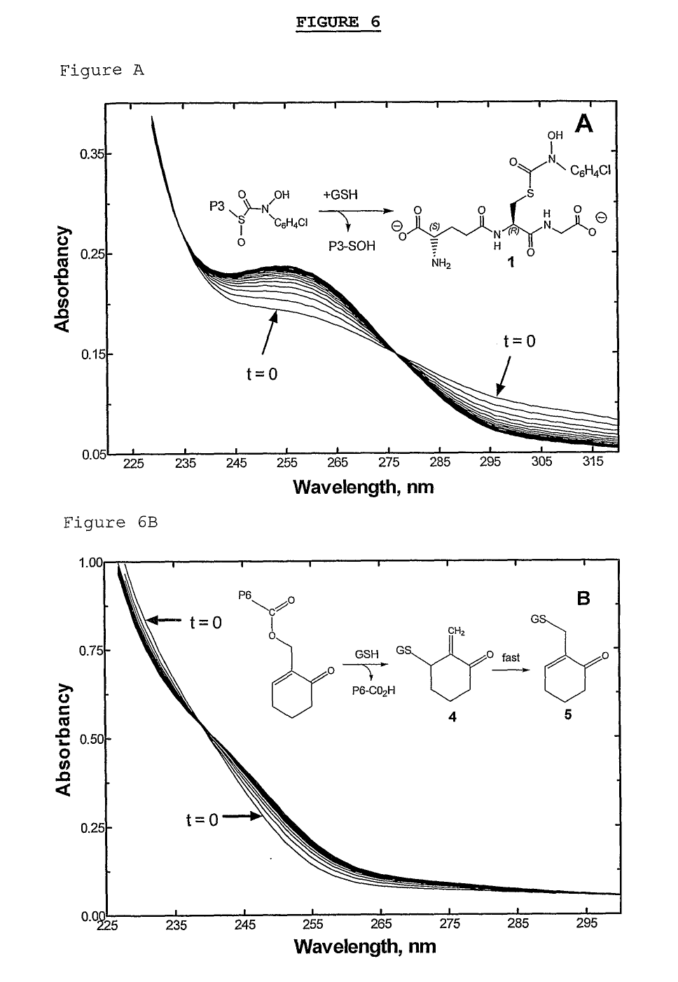

As shown in FIG. 6, the rate constants for formation of these species correlated well with the first-order rate of decrease in absorbancy at 305 nm and 235 nm, corresponding to the loss of the sulfoxide (FIG. 6A) and cyclohexenone functions (FIG. 6B) of the copolymers, respectively. These wavelengths were selected for the kinetic analyses, as they permit the independent assessment of the rates of loss of each functional group in copolymers containing both functional groups.

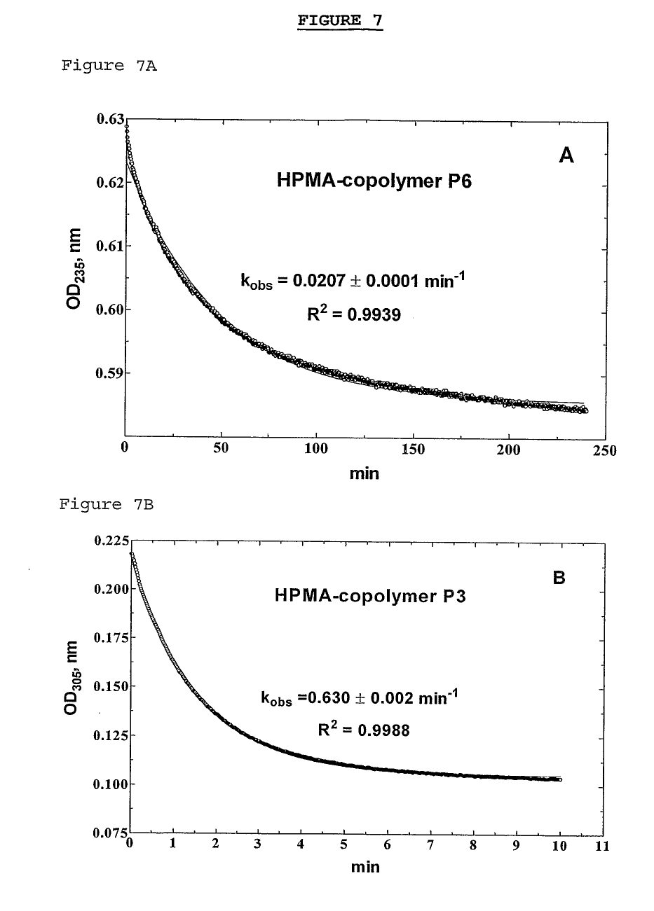

The rates of formation of compounds (5) and (1) from copolymers containing variable amounts of either the cyclohexenyl and/or 8-sulfoxide groups conform to first order kinetics over 4-5 half-lives (FIG. 7).

HPMA copolymers are particularly well designed to serve as platforms for delivering GSH-activated prodrugs into cells via endocytosis. The reaction between excess GSH and the copolymers to form the GlxI inhibitor, i.e. compound (1), or the alkylating agent, i.e. compound (4), follows simple first order kinetic behavior over several half lives (FIG. 7). This indicates that the copolymers exist primarily in an open or extended conformation in solution, which allows free access of GSH to the reactive groups appended to the copolymers. Moreover, the polyacrylamide backbone does not interfere significantly with the reaction between GSH and the 8-sulfoxide function, as the rate constants for reaction of GSH with copolymers P1-P3 (FIG. 4) are at least as large as that reported for the reaction of GSH with the simple sulfoxide derivative compound (2) (Scheme 2): k=1.84±0.07 mM⁻¹min⁻¹, potassium phosphate buffer, 0.1 M (pH 7.5), 25° C. (Hamilton et al, J. Med. Chem. 42:1823-1827 (1999)).

Indicative of a small steric effect on the reaction of GSH with the cyclohexenone functions of P4-P6, the rate constants are about 3-fold smaller than that reported for the reaction of GSH with the crotonate ester, compound (3) of Scheme 3: k=0.068±0.001 mM⁻¹ min⁻¹, potassium phosphate buffer, 0.1 M (pH 6.5), 25° C. (Hamilton et al, Organic Lett. 4:1209-1212 (2002)).

Therefore, the ˜100-fold greater reactivities of the sulfoxide- versus cyclohexenone-containing copolymers reflect primarily the different intrinsic chemical reactivities of the functional groups with GSH, and not the steric properties of the polymer backbone.

Finally, there is little difference in the kinetic properties of the copolymers having either different molecular weights or different mol fractions of the appended sulfoxide or cyclohexenone functions (FIG. 4). Therefore, loading of the HPMA polymer with high levels of prodrug will not adversely affect the kinetic properties of the copolymers. Not surprisingly, the sulfoxide and cyclohexenone functions in the mixed function copolymers P7 and P8 independently react with free GSH, as the rate constants for the individual reactive groups are similar in magnitude to those for the copolymers containing only one of the reactive groups (FIG. 4).

### Example 3

### Stability of Copolymers in Human Serum

To 0.45 mL human serum at 37° C., was added P8 to an initial concentration of approximately 1 mM in 8-sulfoxide and cyclohexenyl groups. As a function of time, 30 μL aliquots of the incubation mixture were transferred to 20 μL of potassium phosphate buffer (0.1 M, pH 7.5) containing 1.6 mM GSH to convert the 8-sulfoxide and cyclohexenyl functions to compounds (1) and (5), respectively. After incubation at room temperature for 5 minutes, the samples were deproteinized by the addition of 100 μL ethanol. The protein precipitate was sedimented by centrifugation at 13,000 g, and the supernatant was fractionated by reverse-phase HPLC, as described in Example 2 above.

The transition state analogue, compound (1), and the GSH adduct, compound (5) were quantified, as described above in Example 2. The rate constants were calculated from the first-order rate of loss of 8-sulfoxide or cyclohexenyl groups as a function of time.

In this manner, the likely chemical stabilities of the 8-sulfoxide and cyclohexenyl groups in the copolymers under physiological conditions were estimated by determining the time-dependent loss of these groups from P8 during incubation with human serum at 37° C. The half-lives for loss of the 8-sulfoxide and cyclohexenyl groups were determined to be 6.6±0.7 and 37.5±7.5 min (n 3), respectively.

The stability of the copolymer prodrugs in circulating human plasma is an important aspect of drug efficacy in humans. The approximate half-lives for the reaction of the sulfoxide- and cyclohexenone-containing copolymers with free GSH (˜2 μM) (Anderson, (1989) supra) that applies in circulating human plasma at pH 7.5 are estimated to be about 175 and 17400 minutes, respectively, using the rate constants in FIG. 4. However, these half-lives are significantly longer than the half-lives of 7 and 38 minutes determined for the loss of the sulfoxide and cyclohexenone functions, respectively, when the copolymers are incubated in noncirculating human plasma.

Therefore, the chemistry associated with the latter process is most significant in determining the bioavailability of the copolymer prodrugs to tumor cells. Compounds with relatively short half-lives in circulating plasma are not preferred candidates for optimizing drug pharmacokinetics via controlled release.

The HPMA copolymer prodrugs undergoing clinical evaluation at this time have half-lives in circulating plasma on the order of hours (Duncan et al, Anti-Cancer Drugs 3:175-210 (1992)). However, continuous infusion might still be used to optimize the plasma pharmacokinetics of less stable copolymer prodrug conjugates and still allow tumor targeting via the EPR effect.

### Example 4

### In Vitro Cytotoxicity Studies

Murine B16 melanotic melanoma was obtained from the DCT Tumor Repository (NCI-Cancer Research and Development Center, Frederick, Md.) and was maintained in RPMI 1640 medium containing L-glutamate (Gibco BRL, Gaithersburg, Md.), supplemented with 10% heat-inactivated fetal calf serum and gentamycin (10 μg/mL), under 37° C. humidified air containing 5% CO₂. Under these conditions B16 cells have a doubling time of about 26 hours. For the toxicity studies, cells in logarithmic growth were introduced into 24 well plates at a density of 2×10⁴ cells/ml in the absence and presence of drug spanning the IC₅₀ values.

After 72 hours, the cells were washed with Hank's balanced salt solution without Ca²⁺, treated with trypsin for 10 minutes at 37° C., concentrated by centrifugation and cell densities determined with a Coulter Counter. Cell viabilities were determined by the trypan blue exclusion method (Kaltenbach et al, Exp. Cell. Res. 15:112-117 (1958)).

Reported IC₅₀ values (mean ±standard deviation of triplicate determinations carried out in three separate assays on different days) were calculated using the Hill equation and the program ADAPT (D'Argenio and Schumitzky, Comput. Methods Programs Biomed. 9:115-134 (1979)).

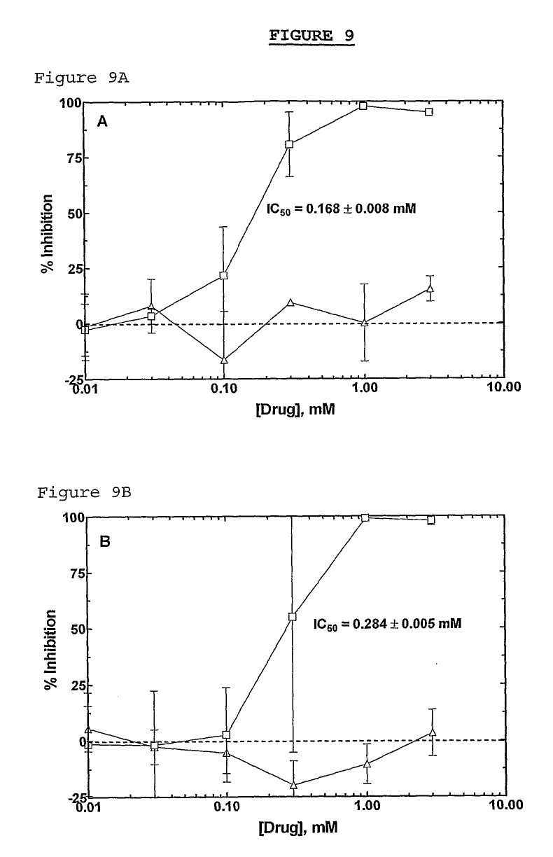

Both the HPMA-8-sulfoxide copolymer and HPMA-7 copolymer inhibit the growth of B16 in a concentration range where the HPMA polymer alone shows no activity (FIGS. 8 and 9). Therefore, growth inhibition must result from the prodrug component of the copolymer.

The simple ethyl, propyl and butyl sulfoxide prodrugs (2, 2a, and 2b) have IC₅₀ values that are roughly five to ten-fold lower than that of HPMA-8-sulfoxide (P1), while the IC₅₀ value of compound (3) is over 7000-fold lower than that of HPMA-7 (P4). In contrast, the IC₅₀ values for P1, P4 and the copolymer containing both prodrugs (P7) differ by no more than a factor of three.

The antitumor activities of the HPMA-copolymer prodrugs listed in FIG. 8 are most likely due to the copolymers themselves and not to toxic contaminants in the copolymer preparations. Antitumor activity is unlikely to arise from unreacted sulfoxide-8 and/or compound (7), as neither NMR nor HPLC analysis of the copolymer preparations indicate that these species are present. Conceivably, compound (1) and/or (4) might form in the growth medium during the efficacy studies, due to reaction of the copolymers with contaminating GSH (perhaps arising from cell lysis). However, this is an unlikely source of antitumor activity, because highly charged S-substituted GSH derivatives like compounds (1) and (4) of FIG. 1 do not readily diffuse across cell membranes near neutral pH (Kavarana et al, J. Med. Chem. 42:221-228 (1999)). Thus, the observed antitumor activities most likely result from processing of the HPMA copolymers inside tumor cells, during or after endocytosis.

The in vitro antitumor activity of sulfoxide copolymer P1 (FIG. 8) implies the presence of intralysosomal GSH to react with P1 to give the cytotoxic transition state analogue inhibitor (1) in FIG. 1. This follows from reports that yeast GlxI is highly specific for GSH and will not use cysteine or cysteinylglycine as cofactors (Kermack and Matheson, Biochem. J. 65:48-58 (1957)). Conceivably, the cysteine and/or cysteinylglycine homologs of compound (1) might form inside the lysosomes, diffuse across the lysosomal membrane into the cytosol and then form (1) via acyl-interchange with cytosolic GSH. This kind of cofactor specificity is not required to explain the cytotoxicity of the cyclohexenone-containing copolymer P7, as intralysosomal GSH, cysteine or cysteinylglycine could all react with P7 to give cytotoxic exocyclic enones.

Both the sulfoxide- and cyclohexenone-containing copolymer prodrugs are significantly less potent than the low molecular weight prodrugs designed to enter cells by diffusion across the cell membrane. HPMA-8 sulfoxide (P1) is about 6.5-fold less potent than sulfoxides 2, 2a, and 2b, and HPMA-7 (P4) is about 7×10³-fold less potent that the simple 2-substituted cyclohexenone, compound 3. This is not a new phenomenon. For example, the HPMA copolymer of 6-(3-aminopropyl)-ellipticine (APE) is >75-fold less potent than APE with B16F10 melanoma in vitro, although the copolymer is significantly more potent in tumor-bearing mice (Searle et al, Bioconjugate Chem. 12:711-718 (2001)). This has been attributed to the slow rate of endocytotic uptake of the copolymer and the slow rate of peptide hydrolysis of the linker, which can have half-lives on the order of many hours.

Likewise, endocytotic uptake of the copolymers described herein might also be a slow process; the rate constants for formation of compounds (1) and (4) of FIG. 1 at pH 5.3 (lysosomes) will be approximately 50-fold smaller that the rate constants that apply in the cytosol with a pH of 7 (cytosol).

Moreover, efficacy might also be limited by the rate of expulsion of the prodrugs from the lysosomes into the cytoplasm of the cell.

Finally, the copolymer prodrugs containing mixed functional groups (for example, copolymers P7 and P8) provide an elegant manner of administering combination chemotherapy.

Any patents or publications referenced in this specification are indicative of the level of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent herein. The present examples along with the methods, procedures, treatments, molecules, and specific compounds described herein and other uses will occur to those skilled in the art that is encompassed within the spirit of the invention as defined by the scope of the claims.

What is claimed is:

1. A prodrug comprising:
a macromolecular carrier; and
at least one precursor of a GlxI inhibitor covalently linked to said macromolecular carrier, wherein said precursor contains a sulfoxide adjacent to an acyl group; and
wherein in the presence of glutathione, an active GlxI inhibitor is formed and released from said carrier as a result of an acyl interchange reaction with the thiol of said glutathione at the acyl group of said precursor.

2. The prodrug of claim 1, wherein said macromolecular carrier has an average molecular mass of from 10 to 50 kDa.

3. The prodrug of claim 1, wherein said macromolecular carrier and said precursor are covalently linked through an amide.

4. The prodrug of claim 1, wherein said macromolecular carrier is a polyacrylamide or polymethacrylamide.

5. The prodrug of claim 4, wherein said polymethacrylamide is poly-N-(2-hydroxypropyl)methacrylamide (HPMA).

6. The prodrug of claim 1, wherein said precursor is an S—(N-aryl/alkyl-N-hydroxycarbamoyl)alkyl sulfoxide.

7. The prodrug of claim 6, wherein said precursor is selected from the group consisting of:
S—(N-p-chlorophenyl-N-hydroxycarbamoyl)ethyl sulfoxide,
S—(N-4-chlorophenyl-N-hydroxycarbamoyl)propyl sulfoxide,
S—(N-4-chlorophenyl-N-hydroxycarbamoyl)butyl sulfoxide,
S—(N-p-bromophenyl-N-hydroxycarbamoyl)ethyl sulfoxide,
S—(N-p-iodophenyl-N-hydroxycarbamoyl)ethyl sulfoxide,
S—(N-phenyl-N-hydroxycarbamoyl)ethyl sulfoxide,
S—(N-methyl-N-hydroxycarbamoyl)ethyl sulfoxide,
S—(N-ethyl-N-hydroxycarbamoyl)ethyl sulfoxide,
S—(N-propyl-N-hydroxycarbamoyl)ethyl sulfoxide,
S—(N-butyl-N-hydroxycarbamoyl)ethyl sulfoxide,
S—(N-pentyl-N-hydroxycarbamoyl)ethyl sulfoxide, and
S—(N-hexyl-N-hydroxycarbamoyl)ethyl sulfoxide.

8. The prodrug of claim 1, wherein said active GlxI inhibitor is selected from the group consisting of:
S—(N-p-chlorophenyl-N-hydroxycarbamoyl)glutathione,
S—(N-p-bromophenyl-N-hydroxycarbamoyl)glutathione,
S—(N-p-iodophenyl-N-hydroxycarbamoyl)glutathione,
S—(N-phenyl-N-hydroxycarbamoyl)glutathione,
S—(N-methyl-N-hydroxycarbamoyl)glutathione,
S—(N-ethyl-N-hydroxycarbamoyl)glutathione,
S—(N-propyl-N-hydroxycarbamoyl)glutathione,
S—(N-butyl-N-hydroxycarbamoyl)glutathione,
S—(N-pentyl-N-hydroxycarbamoyl)glutathione, and
S—(N-hexyl-N-hydroxycarbamoyl)glutathione.

9. The prodrug of claim 5, wherein said precursor is selected from the group consisting of:
S—(N-p-chlorophenyl-N-hydroxycarbamoyl)ethyl sulfoxide,
S—(N-4-chlorophenyl-N-hydroxycarbamoyl)propyl sulfoxide,
S—(N-4-chlorophenyl-N-hydroxycarbamoyl)butyl sulfoxide,
S—(N-p-bromophenyl-N-hydroxycarbamoyl)ethyl sulfoxide,
S—(N-p-iodophenyl-N-hydroxycarbamoyl)ethyl sulfoxide,
S—(N-phenyl-N-hydroxycarbamoyl)ethyl sulfoxide,
S—(N-methyl-N-hydroxycarbamoyl)ethyl sulfoxide,
S—(N-ethyl-N-hydroxycarbamoyl)ethyl sulfoxide,
S—(N-propyl-N-hydroxycarbamoyl)ethyl sulfoxide,
S—(N-butyl-N-hydroxycarbamoyl)ethyl sulfoxide,
S—(N-pentyl-N-hydroxycarbamoyl)ethyl sulfoxide, and
S—(N-hexyl-N-hydroxycarbamoyl)ethyl sulfoxide.

10. The prodrug of claim 1, wherein said prodrug comprises two or more of said precursors, which may be the same or different, covalently linked to said carrier.

11. The prodrug of claim 1, wherein the mol % of said precursor is at least 1.5.

12. The prodrug of claim 1, wherein the mol % of said precursor is at least 8.

13. The prodrug of claim 1, wherein said prodrug further comprises an endocyclic enone covalently linked to said macromolecular carrier, wherein said endocyclic enone forms an active alkylating agent through a Michael addition reaction with the thiol of a glutathione molecule, and wherein said active alkylating agent is released from said carrier as a result of said Michael addition reaction.

14. The prodrug of claim 13, wherein said endocyclic enone is selected from the group consisting of:
2-substituted-2-cyclohexenone,
2-substituted-2-cycloheptenone,
2-substituted-2-cyclopentenone,
2-substituted-benzoquinone,
2-substituted-napthoquinone, and
2-substituted-anthroquinone

15. The prodrug of claim 14, wherein said carrier is poly-N-(2-hydroxypropyl)methacrylamide (HPMA).

16. The prodrug of claim 15, wherein the active GlxI inhibitor is selected from the group consisting of:
S—(N-p-chlorophenyl-N-hydroxycarbamoyl)glutathione,
S—(N-p-bromophenyl-N-hydroxycarbamoyl)glutathione,
S—(N-p-iodophenyl-N-hydroxycarbamoyl)glutathione,
S—(N-phenyl-N-hydroxycarbamoyl)glutathione,
S—(N-methyl-N-hydroxycarbamoyl)glutathione,
S—(N-ethyl-N-hydroxycarbamoyl)glutathione,
S—(N-propyl-N-hydroxycarbamoyl)glutathione,
S—(N-butyl-N-hydroxycarbamoyl)glutathione,
S—(N-pentyl-N-hydroxycarbamoyl)glutathione, and
S—(N-hexyl-N-hydroxycarbamoyl)glutathione.

17. A pharmaceutical composition comprising the prodrug of claim 1 as an active ingredient together with a pharmaceutically acceptable diluent.

18. A pharmaceutical composition comprising the prodrug of claim 13 as an active ingredient together with a pharmaceutically acceptable diluent.

19. A method of treating a subject having a neoplastic condition comprising administering to a subject in need of such treatment a pharmaceutically effective amount of the prodrug of claim 1.

20. A method of treating a subject having a neoplastic condition comprising administering to a subject in need of such treatment a pharmaceutically effective amount of the prodrug of claim 13.

21. The method of claim 19, wherein said pharmaceutically effective amount is from 0.01 g of macromolecular prodrug containing from 8 to 10 mol % precursor to about 1.0 g of macromolecular prodrug containing from 8 to 10 mol % precursor.

22. The method of claim 20, wherein said pharmaceutically effective amount is from 0.01 g of macromolecular prodrug containing from 8 to 10 mol % precursor and alkylating agent combined to about 1.0 g of macromolecular prodrug containing from 8 to 10 mol % precursor and alkylating agent combined.

23. The method of claim 19, wherein said neoplastic condition is selected from the group consisting of breast cancer, ovarian cancer, prostate cancer, lung cancer, colon cancer, kidney cancer, liver cancer, brain cancer, and heamopoetic tissue cancer.

24. The method of claim 20, wherein said neoplastic condition is selected from the group consisting of breast cancer, ovarian cancer, prostate cancer, lung cancer, colon cancer, kidney cancer, liver cancer, brain cancer, and heamopoetic tissue cancer.

25. A method of inhibiting the proliferation of a tumor cell comprising contacting a tumor cell with an amount of the compound of claim 1 effective to inhibit proliferation of said tumor cell.

26. A method of inhibiting the proliferation of a tumor cell comprising contacting a tumor cell with an amount of the compound of claim 13 effective to inhibit proliferation of said tumor cell.

27. A method of forming an active GlxI inhibitor comprising contacting the prodrug of claim 1 with glutathione.

28. A method of forming an active GlxI inhibitor and an alkylating exocyclic enone comprising contacting the prodrug of claim 13 with glutathione.

29. A method for producing a copolymer prodrug comprising:
performing an acyl interchange reaction with S—(N-aryl/alkyl/hydroxy-N-hydroxycarbamoyl)alkyl sulfoxide and a thioamine to form S—(N-aryl/alkyl-N-hydroxycarbamoyl)thioalkylamine;
reacting said S—(N-aryl/alkyl-N-hydroxycarbamoyl)thioalkylamine with methacryloyl chloride and pyridine so as to form S—(N-aryl/alkyl-N-hydroxycarbamoylthioalkyl)methacrylamide; and
co-polymerizing said S—(N-aryl/alkyl-N-hydroxycarbamoylthioalkyl)methacrylamide with an acrylamide to form said copolymer prodrug.

30. The method of claim 29, wherein said thioamine is cysteamine.

31. The method of claim 29, wherein said S—(N-aryl/alkyl-N-hydroxycarbamoylthioalkyl)methacrylamide is copolymerized with HPMA in the presence of azobisisobutylnitrile.

32. A method for producing a copolymer prodrug comprising:
reacting 2-hydroxymethyl-2-endocyclic enone with methacryloyl chloride so as to form 2-methacryloyloxymethyl-2-endocyclic enone, and
co-polymerizing said 2-methacryloyloxymethyl-2-endocyclic enone with an acrylamide to form said copolymer prodrug.

33. The method of claim 32, wherein said 2-methacryloyloxymethyl-2-endocyclic enone is formed in a reaction with 4-methylmorpholine.

34. The method of claim 32, wherein said methacryloyloxymethyl-2-endocyclic enone is copolymerized with HPMA in the presence of azobisisobutylnitrile.

35. The method of claim 27, wherein aryl/alkyl-N-hydroxycarbamoylthioalkyl)methacrylamide is copolymerized with HPMA and methacryloyloxymethyl-2-endocyclic enone in the presence of azobisisobutylnitrile.
